(19) United States
(12) Patent Application Publication
(10) Pub. No.: US 2026/0294942 A1
(43) Pub. Date: Oct. 1, 2026

(54) 3’-FUCOSYLLACTOSE

(71) Applicant: Chr. Hansen A/S, Hoersholm (DK)

(72) Inventor: Stina JENSEN, Hoersholm (DK)

(73) Assignee: Chr. Hansen A/S, Hoersholm (DK)

(21) Appl. No.: 19/476,046

(22) PCT Filed: Apr. 18, 2024
(86) PCT No.: PCT/EP2024/060502

§ 371 Date: Oct. 16, 2025

(30) Foreign Application Priority Data
Apr. 19, 2023 (EP) 23168742.7

(51) Int. Cl. A61K 31/702; A23L 33/00; A61K 35/00; A61K 35/741

(52) U.S. Cl. CPC A61K 31/702; A23L 33/40; A61K 35/741; A61K 2035/115

(57) ABSTRACT

The present disclosure relates to the use of 3-fucosyllactose for modulating inflammation.

### FIELD

The present disclosure relates, in part, to uses of 3-fucosyllactose (3-FL) for modulating alkaline phosphatase (ALP) activity in a subject. Further disclosed herein are methods, uses, processes, and the like.

### BACKGROUND

Human milk oligosaccharides (HMOs) are non-digestible carbohydrates found in human milk. Their importance to infant nutrition is underscored by their position as the third most abundant solid component of human milk, behind lactose and lipids. HMOs can be structurally categorized as (a) fucosylated HMOs such as 2′-and 3-fucosyllactose (2′-FL and 3-FL), (b) neutral non-fucosylated HMOs such as lacto-N-tetraose (LNT) and (c) sialylated HMOs such as 3′- and 6′ sialyllactose (3′-SL and 6′-SL).

HMOs in human milk vary widely based on various influences such as genetics, lactation, and geographic location. While most HMO concentrations decrease over the course of lactation, at least two, 3′-SL and 3-FL, may increase. Different HMOs may work together in complementary ways to support the growth and development of infants. HMOs are thought to have various biological functions, such as preventing the attachment of pathogens to epithelial cells (Ruiz-Palacios, Cervantes, Ramos, Chavez-Munguia, & Newburg, 2003), modulating immune cell responses (Zhang et al., 2019) in vitro, modulating gut microbiota (Berger et al., 2020; Elison et al., 2016; Iribarren et al., 2020). Specific stimulation of gut bacteria by fucosylated HMOs (2′FL, 3FL and DFL) has been seen in an in vitro anaerobic culture system using infant fecal microbiota (Yu et al., 2013).

It has been suggested that HMOs can lower the risk of gut microbiome imbalance due to harmful bacteria (Weichert, Stefan, et al. Nutrition research 10 (2013): 831-838). Further, it has been suggested that HMOs can selectively stimulate helpful bifidobacteria in support of overall gut health (Bode, Lars. Nutrition reviews suppl_2 (2009)), support microbial colonization and gut barrier function (Kong et. al., Mol. Nutr. Food Res. 2019, 63), and enhance mucus barrier function through direct modulation of intestinal goblet cells (Cheng et. al., Mol. Nutr. Food Res. 2020, 64).

Alkaline phosphatase (AP) is a ubiquitous membrane-bound glycoprotein that catalyzes phosphate monoesters' hydrolysis from organic compounds, an essential process in cell signaling. Four AP isozymes have been described in humans, placental AP, germ cell AP, tissue nonspecific AP, and intestinal AP (IAP). IAP seems to play a role in gut microbial homeostasis, nutrient uptake, and inflammation. IAP dysfunction is associated with persistent inflammatory disorders. For example, AP is a strong predictor of mortality in the general population and patients with cardiovascular and chronic kidney disease (CKD). CKD is a disease characterized by gut microbiota imbalance and persistent low-grade inflammation. Mitigating inflammation and dysbiosis can prevent cardiovascular complications in patients with CKD (Alvarenga L. et. al. EPMA J. 2020 Nov. 18; 11(4):565-579).

When a host is attacked by acute pathogens, bacteria may move across the intestinal wall, which is known as gut-origin bacteria translocation (BT). Peritonitis is a commonly observed condition that may cause systemic inflammatory response syndrome, sepsis, and ultimately, multiple organ failure. In patients with peritonitis, not only does the peritoneal cavity act as the source of systemic infection, but the impaired intestinal barrier also facilitates the transmission of gut microbes into the blood stream. These effects are due primarily to changes in the expression of inflammatory factors, such as VEGF, that alter intestinal permeability and intestinal tight junction proteins. Intestinal alkaline phosphatase (IAP or ALP) may reduce peritonitis-related mortality and decrease intestinal permeability. IAP is an alkaline phosphatase isoform that is produced exclusively in the small intestinal mucosa. At the brush edge of the mucosa, IAP may ameliorate the increase in intestinal permeability during peritonitis; thereby, improving the prognosis of peritonitic animals (Wang et. al., PLOS One 2015; 10(5)).

Lipopolysaccharides (LPSs) are bacterial surface glycolipids, produced by Gram-negative bacteria. LPS is known to determine acute inflammatory reactions, particularly in the context of sepsis. However, LPS can also trigger chronic inflammation. In this case, the source of LPS is not an external infection, but rather an increase in endogenous production, which is usually sustained by gut microbiota (GM), and LPS contained in food. The first site in which LPS can exert its inflammatory action is the gut: both GM and gut-associated lymphoid tissue (GALT) are influenced by LPS and shift towards an inflammatory pattern. LPS, particularly when it is chronically present at low levels, has been implicated in a number of conditions such as septic shock and endotoxemia. Metabolic endotoxemia is a condition in which alterations in the gut epithelial barrier allow microbiota-produced LPS to enter the bloodstream and is typically present in inflammatory bowel diseases (IBD). Low-grade inflammation has been linked with many different diseases, such as diabetes, obesity, non-alcoholic fatty liver diseases, chronic kidney disease, and cardiovascular disease.

Modulating ALP activity may be beneficial in a variety of disorders.

### SUMMARY

The present disclosure provides compositions, uses, methods and the like for modulating ALP activity. In particular, the present disclosure relates to 3-FL modulating ALP activity.

The present disclosure provides 3-FL for use in immature individuals (i.e. those less than 3 years of age) for the maturation of the intestinal epithelium, enhancing intestinal barrier integrity, reducing inflammation, modulating LPS, and the like.

While not wishing to be bound by theory, it is believed 3-FL can enhance ALP activity which modulates the level of LPS in the intestine of the subject. This modulation has an anti-inflammatory effect, especially when the subject is experiencing an inflammatory condition.

### BRIEF DESCRIPTION OF THE DRAWINGS

FIG. 1 shows the intestinal alkaline phosphatase level.

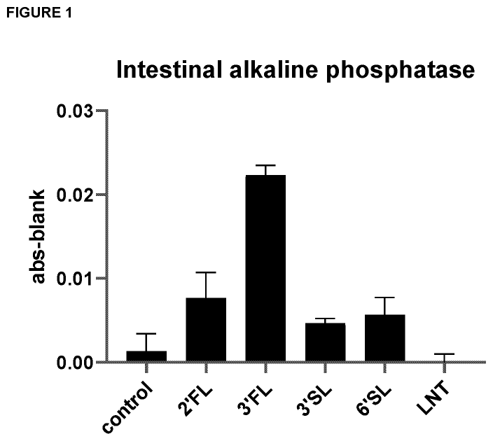

FIG. 2 shows the effect of various HMOs on intestinal barrier integrity in non-challenged (−) and challenged (+) conditions. TEER was measured across Caco-2 cell monolayers exposed for 24 h to HMOs at concentrations of 1, 5 or 20 mg/mL in the absence (top) or presence (bottom) of TNF-α (100 ng/ml) and IFN-γ (10 ng/ml). Data are expressed as means of normalized AUC values+SEM (n=3-9 independent experiments). Statistical significance was determined by two-way ANOVA followed by Dunnett's multiple comparisons test (****p<0.0001, ***p<0.001, **p<0.01 and *p<0.05 compared to the unstimulated control without HMOs).

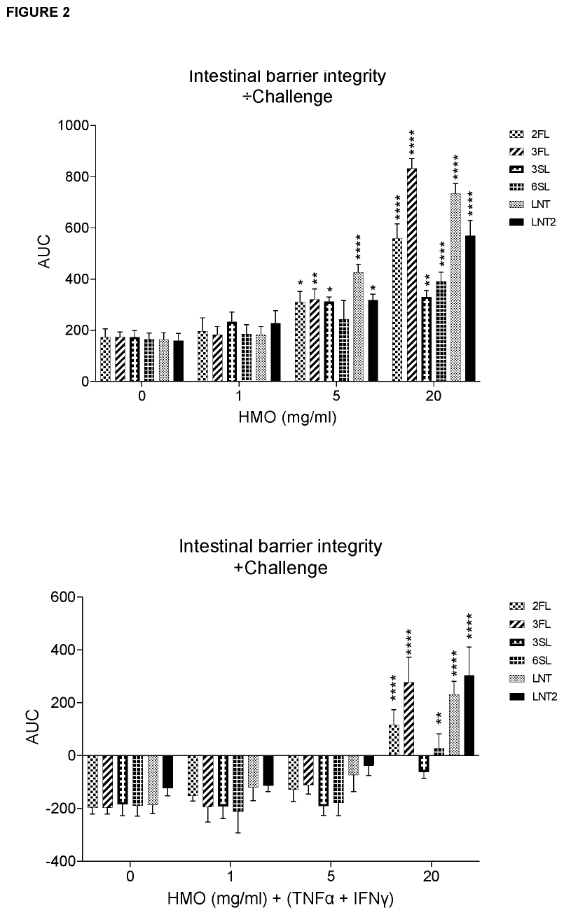

### DETAILED DESCRIPTION

Unless defined otherwise, all technical and scientific terms used herein have the same meaning as commonly understood by persons skilled in the art. Although any methods and materials equivalent or similar to those described herein can be used in the practice of the present disclosure, typical methods and materials are described. All methods described herein can be performed in any suitable order unless otherwise indicated herein or otherwise clearly contradicted by context.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range including the two end values, unless otherwise indicated herein, and each separate value is incorporated into the specification as if it were individually recited herein. The use of any and all examples, or exemplary language (e.g., “such as”) provided herein, is intended merely to better illuminate the invention and does not pose a limitation on the scope of the invention unless otherwise claimed.

As used herein, the term “and/or” is intended to mean the combined (“and”) and the exclusive (“or”) use, i.e. “A and/or B” is intended to mean “A alone, or B alone, or A and B together”.

As used herein the terms “effective amount”, “effective concentration”, or “effective dosage” are defined as the amount, concentration, or dosage of a material sufficient to improve the overall health of the animal and confer benefits similar to the ones demonstrated in the examples. The actual effective dosage in absolute numbers depends on factors including the state of health of the subject in question, and other ingredients present. The “effective amount”, “effective concentration”, or “effective dosage” of the material may be determined by routine assays known to those skilled in the art.

As used herein the term “isolated” means that the bacterial strains described herein are in a form or environment which does not occur in nature, i.e. the strain is at least partially removed from one or more or all of the naturally occurring constituents with which it is associated in nature.

A bacterial “strain” as used herein refers to a bacterium which remains genetically unchanged when grown or multiplied and that originates from a single isolate or pure culture. Probiotics are classified by their genus (e.g. Bifidobacterium), species and subspecies (e.g. animalis subsp. lactis), and strains (e.g. DSM 15954 and/or BB-12®). FAO/WHO has stated that probiotic effects are strain specific and that most probiotic characteristics of a particular strain cannot therefore be extrapolated to other strains of the same species.

As used herein, the term “probiotic” refers to a culture of live or freeze-dried microorganisms, dead microorganisms, fragments of microorganisms and extracts or supernatants of microorganisms which, when applied to man or animal, beneficially affects the host (Hill et al. (2014) Expert Consensus Document, The International Scientific Association for Probiotics and Prebiotics. Consensus statement on the scope and appropriate use of the term probiotic).

The term “human milk oligosaccharide” or “HMO”, as used herein, unless otherwise specified, refers generally to a number of complex carbohydrates found in human breast milk that can be in acidic or neutral form, and to precursors thereof. Exemplary non-limiting human milk oligosaccharides include 3′-sialyllactose, 6′-sialyllactose, 3-fucosyllactose, 2′-fucosyllactose, and lacto-N-neo-tetraose.

The terms “treat” or “treating” should not be taken to imply that an individual is treated until total recovery. Accordingly, these terms broadly include amelioration and/or prevention of the onset of the symptoms or severity of a particular condition.

The term “shelf stable” as used herein, unless otherwise specified, refers to a nutritional product that remains commercially stable after being packaged and then stored at 18-24° C. for at least 3 months, including from about 6 months to about 24 months, and also including from about 12 months to about 18 months.

The terms “nutritional formulation” or “nutritional composition” as used herein, are used interchangeably and, unless otherwise specified, refer to nutritional liquids, nutritional powders, nutritional supplements, and any other nutritional food product as known in the art. The nutritional powders may be reconstituted to form a nutritional liquid, all of which comprise one or more of fat, protein and carbohydrate and are suitable for oral consumption by a human.

The term “nutritional powder” as used herein, unless otherwise specified, refers to nutritional products in flowable or scoopable form that can be reconstituted with water or another aqueous liquid prior to consumption and includes both spray-dried and dry-mixed dry-blended powders.

The term “newborn” as used herein, unless otherwise specified, refers to a person from birth up to four weeks of age. The term “infant” as used herein, unless otherwise specified, refers to a person 12 months or younger. The term “preterm” as used herein, refers to a baby born prior to 36 weeks of gestation. The term “toddler” as used herein, unless otherwise specified, refers to a person greater than one year of age up to three years of age. The term “child” as used herein, unless otherwise specified, refers to a person greater than three years of age up to twelve years of age.

The term “formula” as used herein, unless otherwise specified, refers to liquid and solid human milk replacements or substitutes that are suitable for consumption by a human.

The term “human milk fortifier” as used herein, unless otherwise specified, refers to liquid and solid nutritional products suitable for mixing with breast milk or formula for consumption by a preterm or term infant.

The terms “inflammatory disease” or “inflammatory condition” as used herein, unless otherwise specified, refer to any disease, disorder, or condition characterized by inflammation. The term “infection-mediated inflammatory disease” as used herein, unless otherwise specified, refers to an inflammatory disease associated or induced by microbial infection, including viral and bacterial infection.

The terms “susceptible” and “at risk” as used herein, unless otherwise specified, mean having little resistance to a certain condition or disease, including being genetically predisposed, having a family history of, and/or having symptoms of the condition or disease. The terms “modulating” or “modulation” or “modulate” as used herein, unless otherwise specified, refer to the targeted movement of a selected characteristic.

All percentages, parts and ratios as used herein, are by weight of the total composition, unless otherwise specified. All such weights, as they pertain to listed ingredients, are based on the active level and, therefore, do not include solvents or by-products that may be included in commercially available materials, unless otherwise specified.

Numerical ranges as used herein are intended to include every number and subset of numbers within that range, whether specifically disclosed or not.

The present invention has been described with reference to various embodiments, aspects, examples, or the like. It is not intended that these elements be read in isolation from one another. Thus, the present disclosure provides for the combination of two or more of the embodiments, aspects, examples, or the like.

All embodiments described herein are intended to be within the scope of the invention disclosed. These and other embodiments of the present invention will become readily apparent to those skilled in the art from the following detailed description of the preferred embodiments having reference to the whole description, the invention not being limited to any particular preferred embodiment(s) disclosed. No language in the specification should be construed as indicating any non-claimed element as essential to the practice of the invention.

The present compositions comprise 3-fucosyllactose. The compositions may comprise other Human Milk Oligosaccharide (HMO) such as, for example, 2′-fucosyllactose, 3′-sialyllactose, 6′-sialyllactose, lacto-N-tetraose, lacto-N-neotetraose, and combinations thereof. The HMOs may be in any suitable combination. For example, 3-fucosyllactose may be combined with any one, two, three, four, or five of 2′-fucosyllactose, 3′-sialyllactose, 6′-sialyllactose, lacto-N-tetraose, and lacto-N-neotetraose. In one embodiment, the HMO mix comprises 2′-fucosyllactose, 3-fucosyllactose, 3′-sialyllactose, 6′-sialyllactose, and lacto-N-tetraose.

Formulating compositions with HMOs can be somewhat problematic. It has been found that a more reproducible and consistent composition can be achieved through controlling the particle size distribution (PSD) of the HMO. While not wishing to be bound by theory, it is believed that having a somewhat narrow PSD improves the flowability of the HMO enabling a more effective mixing with the other ingredients. In addition, it is believed that a PSD within a certain range provides a better solubility profile. Particle size of an HMO may be determined using a standard method, such as using a sieve tower, which separates the powder into the different fractions after a defined time with a predefined amplitude. The sieves used in such a method may be sieves which comply with DIN ISO 3310-1.

It is preferred that the 3-fucosyllactose (or other HMO) used in the present compositions have the following particle size characteristics:
Percent through mesh #230 (63 μm)—less than about 20%, less than about 18%, less than about 16%, less than or equal to about 15%.
Percent through mesh #100 (150 μm)—greater than about 75%, greater than about 70%, greater than about 65%, greater than or equal to about 60%.
Percent through mesh #45 (355 μm)—greater than about 95%, greater than about 92%, greater than or equal to about 90%.
Percent Through Mesh #20 (850 μm)—100%.

A preferred composition herein is a nutritional composition such as a formula. The nutritional compositions may be in any product form comprising the ingredients described herein, and which is safe and effective for oral administration. The nutritional compositions may be formulated with optional ingredients such as those described herein.

The nutritional compositions of the present disclosure are preferably formulated as dietary product forms, which are defined herein as those embodiments comprising the ingredients of the present disclosure in a product form that then contains at least one of fat, protein, and carbohydrate, and preferably also contains vitamins, minerals, or combinations thereof.

The nutritional compositions may be formulated with sufficient kinds and amounts of nutrients to provide a sole, primary, or supplemental source of nutrition, or to provide a specialized nutritional product for use in individuals afflicted with specific diseases or conditions or with a targeted nutritional benefit as described below. Specific non-limiting examples of product forms suitable for use 3-FL containing compositions as disclosed herein include, for example, liquid and powdered dietary supplements, liquid and powdered human milk fortifiers, liquid and powdered formula.

Nutritional liquids include both concentrated and ready-to-feed nutritional liquids. These nutritional liquids are most typically formulated as suspensions or emulsions, although other liquid forms are within the scope of the present disclosure.

Nutritional emulsions suitable for use may be aqueous emulsions comprising proteins, fats, and carbohydrates. These emulsions are generally flowable or drinkable liquids at from about 1° C. to about 25° C. and are typically in the form of oil-in-water, water-in-oil, or complex aqueous emulsions, although such emulsions are most typically in the form of oil-in-water emulsions having a continuous aqueous phase and a discontinuous oil phase.

The nutritional emulsions may be and typically are shelf stable. The nutritional emulsions typically contain up to about 95% by weight of water, including from about 50% to about 95%, also including from about 60% to about 90%, and also including from about 70% to about 85%, of water by weight of the nutritional emulsions. The nutritional emulsions may have a variety of product densities, but most typically have a density greater than about 1 g/mL, including greater than about 1.05 g/mL, including greater than about 1.055 g/mL to about 1.12 g/mL, and also including from about 1.085 g/mL to about 1.10 g/mL. The nutritional emulsions may have a caloric density tailored to the nutritional needs of the ultimate user, although in most instances the emulsions comprise generally at least 660 kcal/liter, about 675 kcal/liter to about 820 kcal/liter, about 680 kcal/liter to about 800 kcal/liter. In some embodiments, the emulsion may have a caloric density of from about 50-100 kcal/liter to about 660 kcal/liter, including from about 150 kcal/liter to about 500 kcal/liter. In some specific embodiments, the emulsion may have a caloric density of 25, or 50, or 75, or 100 kcal/liter. The nutritional emulsion may have a pH ranging from about 3.5 to about 8, from about 4.5 to about 7.5, including from about 5.5 to about 7.3, including from about 6.2 to about 7.2. Although the serving size for the nutritional emulsion can vary depending upon a number of variables, a typical serving size is generally at least 1 mL, or even at least 2 mL, or even at least 5 mL, or even at least 10 mL, or even at least 25 mL, including ranges from about 1 mL to about 300 mL, including from about 4 mL to about 250 mL, and including from about 10 mL to about 240 mL.

The nutritional solids may be in any solid form but are typically in the form of flowable or substantially flowable particulate compositions, or at least particulate compositions, that may optionally be compressed into tablets. Particularly suitable nutritional solid product forms include spray dried, agglomerated and/or dry-blended powder compositions. The compositions can easily be scooped and measured with a spoon or similar other device, and can easily be reconstituted by the intended user with a suitable aqueous liquid, typically water, to form a nutritional composition for immediate oral or enteral use. In this context, “immediate” use generally means within about 48 hours, most typically within about 24 hours, preferably right after reconstitution. The nutritional powders may be reconstituted with water prior to use to a caloric density tailored to the nutritional needs of the ultimate user, although in most instances the powders are reconstituted with water to form compositions comprising generally at least 660 kcal/liter, about 675 kcal/liter to about 820 kcal/liter, about 680 kcal/liter to about 800 kcal/liter. In some embodiments, the reconstituted powder may have a caloric density of from about 50-100 kcal/liter to about 660 kcal/liter, including from about 150 kcal/liter to about 500 kcal/liter. In some specific embodiments, the reconstituted powder may have a caloric density of 25, or 50, or 75, or 100 kcal/liter.

The present compositions may be useful for avoiding, ameliorating, or otherwise modulating inflammation-related conditions. For example, the present composition may be useful in newborns, infants, toddlers, or children. The present compositions may be useful in reducing the incidence of inflammatory conditions in newborns, infants, toddlers, children. Examples of inflammation-related conditions include allergies, respiratory infections, and chronic inflammation (e.g., irritable bowel disorder (IBD), Crohn's, ulcerative colitis, CKD, and the like).

The present composition may comprise 3-fucosyllactose in any suitable amount, such as, for example, at least 0.001 mg/mL, including from about 0.001 mg/mL to about 20 mg/mL, including from about 0.01 mg/mL to about 10 mg/mL, including from about 0.01 mg/mL to about 5 mg/mL (mg of HMO per mL of composition).

Where the composition is a nutritional powder, the concentration of 3-FL in the nutritional powder is preferably from about 0.001% to about 5%, including from about 0.01% to about 1% (by weight of the nutritional powder). Where the composition is a ready-to-feed nutritional liquid, the concentration of 3-FL is preferably from about 0.001% to about 0.50%, including from about 0.001% to about 0.15%), including from about 0.01% to about 0.10%, and further including from about 0.01%) to about 0.03% (by weight of the ready-to-feed nutritional liquid). Where the composition is a concentrated nutritional liquid, the concentration of 3-FL is preferably from about 0.002% to about 0.6%, including from about 0.002% to about 0.3%, including from about 0.02% to about 0.20% (by weight of the concentrated nutritional liquid).

The compositions of the present disclosure may optionally include anti-inflammatoirech as long-chain polyunsaturated fatty acids (LCPUFAs) and/or antioxidants such as carotenoids. LCPUFAs may be included in the compositions to provide nutritional support and to enhance growth and functional development of the intestinal epithelium and associated immune cell populations. Exemplary LCPUFAs for use in the present compositions include, for example, docosahexaenoic acid (DHA), eicosapentaenoic acid (EPA), arachidonic acid (ARA), linoleic acid, linolenic acid (alpha linolenic acid) and gamma-linolenic acid derived from oil sources such as plant oils, marine plankton, fungal oils, and fish oils. The present compositions preferably comprise total concentrations of LCPUFA of from about 0.01 mM to about 10 mM and including from about 0.01 mM to about 1 mM. Alternatively, the compositions comprise total concentrations of LCPUFA of from about 0.001 g/L to about 1 g L.

Additionally, antioxidants such as carotenoids, and particularly, combinations of the carotenoids, lutein, lycopene, zeaxanthin and/or beta-carotene may be included in the present compositions.

The compositions of the present disclosure may further comprise other optional components that may modify the physical, chemical, aesthetic or processing characteristics of the composition or to serve as pharmaceutical or additional nutritional components. Non-limiting examples of such optional ingredients include preservatives, emulsifying agents, buffers, pharmaceutical actives, nutrients, colorants, flavors, thickening agents and stabilizers, flowing agents, minerals, emulsifying agents, lubricants, sweetening agents, and the like.

A flowing agent or anti-caking agent may be included in the present compositions to retard clumping or caking of the powder over time and to make a powder embodiment flow easily from its container. Non-limiting examples include tricalcium phosphate, silicates, and combinations thereof. The concentration of the flowing agent or anti-caking agent in the nutritional composition varies depending upon the product form, the other selected ingredients, the desired flow properties, and so forth, but most typically range from about 0.1% to about 4%, including from about 0.5% to about 2%, by weight of the nutritional composition.

The compositions of the present disclosure may be prepared by any known or otherwise effective manufacturing technique for preparing the selected product solid or liquid form. Many such techniques are known for any given product form such as nutritional liquids or powders and can easily be applied by one of ordinary skill in the art to the nutritional compositions described herein.

Those skilled in the art will appreciate that the administration of compositions disclosed herein can be carried out with dose levels and dosing regimens as required depending on the circumstances and on the condition of the subject. Suitable dosage regimes can be determined based on the teaching of the present application. Dosage regimens may be adjusted to provide the optimal support of the subject. It will be appreciated that the exact amounts and rates of administration will depend on a number of factors such as the age, body weight, general health, sex, and dietary requirements of the subject. Based on the teaching herein those skilled in the art can, by routine trial and experimentation, determine suitable dosage regimes on a case-by-case basis.

The present compositions may comprise at least one probiotic strain, for example, Lactococcus lactis subsp. lactis biovar. diacetylactis, Lactococcus lactis subsp. cremoris, Lactococcus lactis subsp. lactis, any strain belonging to the genus Lactobacillus (including but not limited to Lactobacillus acidophilus, Lactobacillus casei subsp. casei, Lactobacillus delbrueckii subsp. bulgaricus, Lactobacillus fermentum, Lactobacillus gasseri, Lactobacillus helveticus, Lactobacillus lactis, Lactobacillus rhamnosus, Lactobacillus salivarius), any strain belonging to the genus Bifidobacterium (including but not limited to Bifidobacterium adolescentis, Bifidobacterium angulatum, Bifidobacterium animalis subsp. lactis, Bifidobacterium bifidum, Bifidobacterium breve, Bifidobacterium catenulatum, Bifidobacterium dentium, Bifidobacterium longum subsp. infantis, Bifidobacterium longum subsp. longum, Bifidobacterium magnum, Bifidobacterium pseudocatenulatum), or any strain from the genera of Akkermansia, Anaerostipes, Butyricicoccus, Christensenella, Clostridia, Coprococcus, Dorea, Eubacterium, Faecalibacterium or Roseburia or the family Coriobacteriaceae, as well as suitable combinations of the foregoing.

The present compositions may comprise at least one strain of a bacterium selected from the group comprising Bifidobacterium animalis subsp. lactis deposited as DSM 15954, Lactobacillus acidophilus deposited as DSM 13241, Lactobacillus rhamnosus deposited as ATCC 53103, Lactobacillus paracasei subsp. paracasei deposited as ATCC 55544, Lactobacillus paracasei deposited as LMG-17806, Streptococcus thermophilus deposited as DSM 15957, Lactobacillus fermentum deposited as NM 02/31074, Lactobacillus paracasei subsp. paracasei deposited as CCTCC M204012 and suitable combinations thereof.

The present compositions preferably comprise an effective amount of probiotic. For example, where present it is preferred the probiotic has a concentration ranging from 0.05×10⁹ CFU/g to 30×10⁹ CFU/g, preferably from 0.5×10⁹ CFU/g to 25×10⁹ CFU/g.

### EXAMPLES

Intestinal alkaline phosphatase (iALP) was measured in Caco-2 cells (DSMZ ACC 169), which is a human colorectal adenocarcinoma cell line with characteristics of small intestinal enterocytes as they develop microvilli and form tight junctions. Caco-2 cells were cultured in Gibco™ MEM, GlutaMAX™ Supplement (Gibco™ 41090036) added 10% FBS (Gibco™ 10500064), 1% Pen/strep (Gibco™ 15140122) and 1% Non-Essential Amino Acids Solution (cat #M 7145-100ML). Cells were seeded in 24-well plates (Cat no. 142475) at a concentration of 5E+05 cells/ml. Cells were seeded with 10 mg/ml of single HMOs (2′-FL, 3-FL, 3′-SL, 6′-SL and LNT) for 3-4 days at which timepoint the ALP was measured by QUANTI-Blue (QBS-4313 Cat: rep-qbs). according to manufactures instructions. Data was plotted as absorbance with media blank subtracted.

Results can be seen in the Figures.

Intestinal barrier integrity was measured by trans-epithelial electrical resistance (TEER) for 24 h in fully differentiated Caco-2 cells (3 wks old). Caco-2 (DSMZ ACC 169) was cultured as described above and was seeded at a concentration of 2E+05 cells/well in Corning® Transwell® plates (cat #CLS 3460-48EA) for 3 wks before transferred to CellZscope (NanoAnalytics) to measure the TEER upon stimulation with apical and basolateral HMOs (2′-FL, 3-FL, 3′-SL, 6′-SL, LNT and LNT2) at concentrations from 1 mg/ml to 20 mg/ml for 24h. In some cases, cells were simultaneously exposed to an inflammatory condition of basolateral TNFalpha (SRP3177) at 100 ng/ml and basolateral IFNgamma (cat. #rcyec-hifng) at 10 ng/ml The fucosylated HMOs (2′FL and 3FL) as well as the neutral non-fucosylated HMOS (LNT and LNT2) had the most pronounced effect on TEER at 20 mg/mL. The two sialylated HMOs (3′SL and 6′SL) were less potent. In the presence of an inflammatory condition induced by TNF-α and IFN-γ, the TEER declined after 24 h to ~30% below baseline, which indicates a leakier cell layer. However, the TEER was stabilized to above baseline in the presence of the pro-inflammatory cytokines plus 20 mg/mL HMOs. The effect was again most notable for the fucosylated and neutral non-fucosylated HMOs.

1. A method of modulating inflammation in a newborn, infant, or toddler, said method comprising administering to said subject a composition comprising 3-fucosyllactose.

2. The method of claim 1, wherein the composition is a nutritional composition.

3. The method of claim 2, wherein the composition is a powder.

4. The method of claim 3, where the composition comprises at least 0.01% of 3-fucosyllactose.

5. The method of claim 3, wherein the 3-fucosyllactose has particle size distribution, as measured using sieves complying to DIN ISO 3310-1, of less than about 20% through mesh #230 (63 μm), greater than about 65% through mesh #100 (150 μm), greater than about 92% through mesh #45 (355 μm), and 100% through mesh #20 (850 μm).

6. The method of claim 1, wherein the composition comprises at least 0.5×10⁹ CFU/g of a probiotic.

7. The method of claim 1, wherein the subject is a newborn.

8. The method of claim 1, wherein the subject is an infant.

9. The method of claim 1, wherein the subject is a toddler.
